SEQUENCE LISTING (26 sequences)

SEQ ID NO: 1 - RNA, 25 nt, Homo sapiens
  misc_feature 1..25: note = hs.Ri.FGA.13.5 sense
  source 1..25: mol_type = other RNA
gctctgtatctggtagtactggaca

SEQ ID NO: 2 - RNA, 27 nt, Homo sapiens
  misc_feature 1..27: note = hs.Ri.FGA.13.5 antisense
  source 1..27: mol_type = other RNA
tgtccagtactaccagatacagagctc

SEQ ID NO: 3 - RNA, 25 nt, Homo sapiens
  misc_feature 1..25: note = CD.Ri.281933.13.5 sense
  source 1..25: mol_type = other RNA
ctgatggtcacaaagaagttaccaa

SEQ ID NO: 4 - RNA, 27 nt, Homo sapiens
  misc_feature 1..27: note = CD.Ri.281933.13.5 antisense
  source 1..27: mol_type = other RNA
ttggtaacttctttgtgaccatcagga

SEQ ID NO: 5 - RNA, 25 nt, Homo sapiens
  misc_feature 1..25: note = hs.Ri.FGA.13.8 sense
  source 1..25: mol_type = other RNA
agggttgattgatgaagtcaatcaa

SEQ ID NO: 6 - RNA, 27 nt, Homo sapiens
  misc_feature 1..27: note = hs.Ri.FGA.13.8 antisense
  source 1..27: mol_type = other RNA
ttgattgacttcatcaatcaacccttt

SEQ ID NO: 7 - RNA, 25 nt, Homo sapiens
  misc_feature 1..25: note = hs.Ri.FGA.13.4 sense
  source 1..25: mol_type = other RNA
ggtggacattgatattaagatccga

SEQ ID NO: 8 - RNA, 27 nt, Homo sapiens
  misc_feature 1..27: note = hs.Ri.FGA.13.4 antisense
  source 1..27: mol_type = other RNA
tcggatcttaatatcaatgtccacctc

SEQ ID NO: 9 - RNA, 25 nt, Homo sapiens
  misc_feature 1..25: note = hs.Ri.FGA.13.7 sense
  source 1..25: mol_type = other RNA
atagtggtgaaggtgactttctagc

SEQ ID NO: 10 - RNA, 27 nt, Homo sapiens
  misc_feature 1..27: note = hs.Ri.FGA.13.7 antisense
  source 1..27: mol_type = other RNA
gctagaaagtcaccttcaccactatct

SEQ ID NO: 11 - RNA, 25 nt, synthetic construct
  misc_feature 1..25: note = ms.FGA.1 sense
  modified_base 1: mod_base = cm
  modified_base 2: mod_base = OTHER; note = 2'-O-methyladenosine
  modified_base 4: mod_base = cm
  modified_base 6: mod_base = OTHER; note = 2'-O-methyladenosine
  modified_base 12: mod_base = um
  modified_base 14: mod_base = OTHER; note = 2'-O-methyladenosine
  modified_base 16: mod_base = OTHER; note = 2'-O-methyladenosine
  modified_base 18: mod_base = OTHER; note = 2'-O-methyladenosine
  modified_base 24: mod_base = um
  source 1..25: mol_type = other RNA
caaccaggattttacaaacagaatc

SEQ ID NO: 12 - RNA, 27 nt, synthetic construct
  misc_feature 1..27: note = ms.FGA.1 antisense
  modified_base 3: mod_base = um
  modified_base 9: mod_base = um
  modified_base 11: mod_base = gm
  modified_base 13: mod_base = OTHER; note = 2'-O-methyladenosine
  modified_base 23: mod_base = um
  modified_base 25: mod_base = gm
  modified_base 26: mod_base = gm
  modified_base 27: mod_base = cm
  source 1..27: mol_type = other RNA
gattctgtttgtaaaatcctggttggc

SEQ ID NO: 13 - RNA, 25 nt, synthetic construct
  misc_feature 1..25: note = ms.FGA.2 sense
  modified_base 1: mod_base = gm
  modified_base 2: mod_base = cm
  modified_base 4: mod_base = gm
  modified_base 6: mod_base = OTHER; note = 2'-O-methyladenosine
  modified_base 12: mod_base = um
  modified_base 14: mod_base = OTHER; note = 2'-O-methyladenosine
  modified_base 16: mod_base = OTHER; note = 2'-O-methyladenosine
  modified_base 24: mod_base = OTHER; note = 2'-O-methyladenosine
  source 1..25: mol_type = other RNA
  modified_base 18: mod_base = OTHER; note = 2'-O-methyladenosine
gctgtaaaccgtgagataaatctac

SEQ ID NO: 14 - RNA, 27 nt, synthetic construct
  misc_feature 1..27: note = ms.FGA.2 antisense
  modified_base 3: mod_base = OTHER; note = 2'-O-methyladenosine
  modified_base 9: mod_base = OTHER; note = 2'-O-methyladenosine
  modified_base 11: mod_base = cm
  modified_base 13: mod_base = cm
  modified_base 23: mod_base = OTHER; note = 2'-O-methyladenosine
  modified_base 25: mod_base = cm
  modified_base 26: mod_base = cm
  modified_base 27: mod_base = cm
  source 1..27: mol_type = other RNA
gtagatttatctcacggtttacagccc

SEQ ID NO: 15 - RNA, 25 nt, synthetic construct
  misc_feature 1..25: note = ms.FGA.3 sense
  modified_base 1: mod_base = cm
  modified_base 2: mod_base = OTHER; note = 2'-O-methyladenosine
  modified_base 4: mod_base = gm
  modified_base 6: mod_base = cm
  modified_base 12: mod_base = um
  modified_base 14: mod_base = OTHER; note = 2'-O-methyladenosine
  modified_base 16: mod_base = gm
  modified_base 18: mod_base = OTHER; note = 2'-O-methyladenosine
  modified_base 24: mod_base = um
  source 1..25: mol_type = other RNA
caggtcatcgctaaagaattgcttc

SEQ ID NO: 16 - RNA, 27 nt, synthetic construct
  misc_feature 1..27: note = ms.FGA.3 antisense
  modified_base 3: mod_base = OTHER; note = 2'-O-methyladenosine
  modified_base 9: mod_base = um
  modified_base 11: mod_base = um
  modified_base 13: mod_base = um
  modified_base 23: mod_base = cm
  modified_base 25: mod_base = gm
  modified_base 26: mod_base = um
  modified_base 27: mod_base = um
  source 1..27: mol_type = other RNA
gaagcaattctttagcgatgacctgtt

SEQ ID NO: 17 - RNA, 25 nt, synthetic construct
  misc_feature 1..25: note = hs.Ri.FGA.13.5 methylated sense
  modified_base 1: mod_base = gm
  modified_base 2: mod_base = cm
  modified_base 4: mod_base = cm
  modified_base 6: mod_base = gm
  modified_base 12: mod_base = gm
  modified_base 14: mod_base = um
  modified_base 16: mod_base = gm
  modified_base 18: mod_base = OTHER; note = 2'-O-methyladenosine
  modified_base 24: mod_base = cm
  source 1..25: mol_type = other RNA
gctctgtatctggtagtactggaca

SEQ ID NO: 18 - RNA, 27 nt, synthetic construct
  misc_feature 1..27: note = hs.Ri.FGA.13.5 methylated anti-sense
  modified_base 3: mod_base = um
  modified_base 9: mod_base = OTHER; note = 2'-O-methyladenosine
  modified_base 11: mod_base = um
  modified_base 13: mod_base = cm
  modified_base 23: mod_base = OTHER; note = 2'-O-methyladenosine
  modified_base 25: mod_base = cm
  modified_base 26: mod_base = um
  modified_base 27: mod_base = cm
  source 1..27: mol_type = other RNA
tgtccagtactaccagatacagagctc

SEQ ID NO: 19 - RNA, 25 nt, synthetic construct
  misc_feature 1..25: note = CD.Ri.281933.13.5 methylated sense
  modified_base 1: mod_base = cm
  modified_base 2: mod_base = um
  modified_base 4: mod_base = OTHER; note = 2'-O-methyladenosine
  modified_base 6: mod_base = gm
  modified_base 12: mod_base = OTHER; note = 2'-O-methyladenosine
  modified_base 14: mod_base = OTHER; note = 2'-O-methyladenosine
  modified_base 16: mod_base = OTHER; note = 2'-O-methyladenosine
  modified_base 18: mod_base = gm
  modified_base 24: mod_base = OTHER; note = 2'-O-methyladenosine
  source 1..25: mol_type = other RNA
ctgatggtcacaaagaagttaccaa

SEQ ID NO: 20 - RNA, 27 nt, synthetic construct
  misc_feature 1..27: note = CD.Ri.281933.13.5 methylated antisense
  modified_base 3: mod_base = gm
  modified_base 9: mod_base = um
  modified_base 11: mod_base = cm
  modified_base 13: mod_base = um
  modified_base 23: mod_base = cm
  modified_base 25: mod_base = gm
  modified_base 26: mod_base = gm
  modified_base 27: mod_base = OTHER; note = 2'-O-methyladenosine
  source 1..27: mol_type = other RNA
ttggtaacttctttgtgaccatcagga

SEQ ID NO: 21 - RNA, 25 nt, synthetic construct
  misc_feature 1..25: note = h s.Ri.FGA.13.8 methylated sense
  modified_base 1: mod_base = OTHER; note = 2'-O-methyladenosine
  modified_base 2: mod_base = gm
  modified_base 4: mod_base = gm
  modified_base 6: mod_base = um
  modified_base 12: mod_base = OTHER; note = 2'-O-methyladenosine
  modified_base 14: mod_base = gm
  modified_base 16: mod_base = OTHER; note = 2'-O-methyladenosine
  modified_base 18: mod_base = um
  modified_base 24: mod_base = OTHER; note = 2'-O-methyladenosine
  source 1..25: mol_type = other RNA
agggttgattgatgaagtcaatcaa

SEQ ID NO: 22 - RNA, 27 nt, synthetic construct
  misc_feature 1..27: note = h s.Ri.FGA.13.8 methylated antisense
  modified_base 3: mod_base = gm
  modified_base 9: mod_base = cm
  modified_base 11: mod_base = um
  modified_base 13: mod_base = OTHER; note = 2'-O-methyladenosine
  modified_base 23: mod_base = cm
  modified_base 25: mod_base = um
  modified_base 26: mod_base = um
  modified_base 27: mod_base = um
  source 1..27: mol_type = other RNA
ttgattgacttcatcaatcaacccttt

SEQ ID NO: 23 - RNA, 25 nt, synthetic construct
  misc_feature 1..25: note = hs.Ri.FGA.13.4 methylated sense
  source 1..25: mol_type = other RNA
  modified_base 1: mod_base = gm
  modified_base 2: mod_base = gm
  modified_base 4: mod_base = gm
  modified_base 6: mod_base = OTHER; note = 2'-O-methyladenosine
  modified_base 12: mod_base = OTHER; note = 2'-O-methyladenosine
  modified_base 14: mod_base = OTHER; note = 2'-O-methyladenosine
  modified_base 16: mod_base = um
  modified_base 18: mod_base = OTHER; note = 2'-O-methyladenosine
  modified_base 24: mod_base = gm
ggtggacattgatattaagatccga

SEQ ID NO: 24 - RNA, 27 nt, synthetic construct
  misc_feature 1..27: note = hs.Ri.FGA.13.4 methylated antisense
  modified_base 3: mod_base = gm
  modified_base 9: mod_base = um
  modified_base 11: mod_base = OTHER; note = 2'-O-methyladenosine
  modified_base 13: mod_base = OTHER; note = 2'-O-methyladenosine
  modified_base 23: mod_base = OTHER; note = 2'-O-methyladenosine
  modified_base 25: mod_base = cm
  modified_base 26: mod_base = um
  modified_base 27: mod_base = cm
  source 1..27: mol_type = other RNA
tcggatcttaatatcaatgtccacctc

SEQ ID NO: 25 - RNA, 25 nt, synthetic construct
  misc_feature 1..25: note = hs.Ri.FGA.13.7 methylated sense
  modified_base 1: mod_base = OTHER; note = 2'-O-methyladenosine
  modified_base 2: mod_base = um
  modified_base 4: mod_base = gm
  modified_base 6: mod_base = gm
  modified_base 12: mod_base = gm
  modified_base 14: mod_base = um
  modified_base 16: mod_base = OTHER; note = 2'-O-methyladenosine
  modified_base 18: mod_base = um
  modified_base 24: mod_base = gm
  source 1..25: mol_type = other RNA
atagtggtgaaggtgactttctagc

SEQ ID NO: 26 - RNA, 27 nt, synthetic construct
  misc_feature 1..27: note = hs.Ri.FGA.13.7 methylated antisense
  modified_base 3: mod_base = um
  modified_base 9: mod_base = gm
  modified_base 11: mod_base = cm
  modified_base 13: mod_base = cm
  modified_base 23: mod_base = um
  modified_base 25: mod_base = um
  modified_base 26: mod_base = cm
  modified_base 27: mod_base = um
  source 1..27: mol_type = other RNA
gctagaaagtcaccttcaccactatct

(19) United States
(12) Patent Application Publication
(10) Pub. No.: US 2025/0319023 A1
(43) Pub. Date: Oct. 16, 2025

(54) METHODS AND COMPOSITIONS FOR MODULATING FIBRINOGEN

(71) Applicant: THE UNIVERSITY OF BRITISH COLUMBIA, Vancouver (CA)

(72) Inventors: Christian J. KASTRUP, Vancouver (CA); Lih Jiin JUANG, Vancouver (CA); Amy Wong STRILCHUK, Vancouver (CA); Jerry LEUNG, Vancouver (CA); Madelaine ROBERTSON, Vancouver (CA); Pieter R. CULLIS, Vancouver (CA)

(73) Assignee: THE UNIVERSITY OF BRITISH COLUMBIA, Vancouver, BC (CA)

(21) Appl. No.: 18/712,070

(22) PCT Filed: Nov. 17, 2022
(86) PCT No.: PCT/CA2022/051702

§ 371 Date: May 21, 2024

Related U.S. Application Data
(60) Provisional application No. 63/282,241, filed on Nov. 23, 2021.

(51) Int. Cl. A61K 9/127; A61K 47/10; A61K 47/28; C12N 15/113

(52) U.S. Cl. CPC A61K 9/127; A61K 47/10; A61K 47/28; C12N 15/113; C12N 2310/14

(57) ABSTRACT

The present disclosure provides a lipid nanoparticle comprising an siRNA molecule against fibrinogen alpha chain, the siRNA molecule containing modified or unmodified nucleotides. Further provided is an siRNA molecule against fibrinogen alpha chain, the siRNA molecule containing modified or unmodified nucleotides and is between 15 and 35 nucleotides in length and has at least 80% sequence identity to SEQ ID NOs: 1-10.

### CROSS REFERENCE TO RELATED APPLICATION

This application claims the benefit of U.S. Provisional Patent Application Ser. No. 63/282,241 filed 23 Nov. 2021, entitled “LIPID NANOPARTICLE DELIVERY OF SIRNA”.

### STATEMENT REGARDING SEQUENCE LISTING

This application contains a sequence listing, which has been filed electronically in XML format and is hereby incorporated by reference herein in its entirety. The name of the XML file containing the sequence listing is 1576-P17US.PNP_Sequence_Listing.xml. The XML file is 88.7 KB; was created on 24 Mar. 2025; and is being submitted electronically via Patent Center with the filing of the specification.

### TECHNICAL FIELD

The present disclosure relates to nucleic acid for targeting fibrinogen and pharmaceutical formulations thereof.

### BACKGROUND

Fibrinogen is synthesized by the liver and circulates in plasma at a concentration of 2-4 g/L, with a half-life of 3-5 days in plasma. Fibrinogen contributes to multiple pathologies by modifying inflammatory and malignant processes. The hepatic expression of fibrinogen is significantly upregulated during acute phase response to inflammatory challenges, such as in COVID-19, cancer, and sepsis, and obesity. While fibrinogen is essential for hemostasis, elevated fibrinogen (hyperfibrinogenemia) is a risk factor for thrombosis by causing increased blood viscosity and resistance to fibrinolysis. Indeed, thrombosis is a major cause of death. It is the underlying pathology for many cardiovascular events and is the second leading cause of death in cancer patients. Thrombosis frequently occurs when blood contacts external medical devices, such as dialysis and extracorporeal membrane oxygenation (ECMO) machines, leading to device failure, thereby elevating patient risk. Thrombosis also occurs during severe inflammation (thromboinflammation) caused by increased synthesis of coagulation proteins, downregulation of anticoagulation, and inhibition of fibrinolysis. Additionally, fibrin (ogen) contributes to the metastasis of tumour cells by inhibiting the activity of natural killer cells.

Decreasing circulating fibrinogen levels could attenuate both inflammation and thrombosis, but current agents cannot safely decrease the concentration of fibrinogen for long durations. Fibrinogen-depleting proteases, isolated from snake venom, have been used for reperfusion therapy occasionally, but have short half-lives, and mixed results in their ability to knock down fibrinogen, improve functional outcomes, and prevent recurrence of thrombosis. In addition, resistance to fibrinogen-depleting activity after repeated infusions of the proteases have been reported. Single-stranded antisense oligonucleotides (ASOs) have been used to reduce the concentration of fibrinogen in mice in vivo. However, in general, development of ASOs for clinical use has faced challenges such as liver and kidney toxicity, and severe thrombocytopenia. In vitro studies suggest that small interfering RNA (siRNA) are more effective than ASOs at silencing the expression of target proteins. However, the use of siRNA sequences can cause complete knock-down of target proteins. While this may be desirable for certain disease indications, full knock-down of fibrinogen can compromise hemostasis.

The present disclosure addresses one or more problems described in the prior art and/or provides useful alternatives to known approaches to reduce levels of fibrinogen and/or fibrin.

### SUMMARY

The present disclosure in some embodiments provides a lipid nanoparticle (LNP) comprising siRNA for modifying the expression of fibrinogen, thereby treating and/or preventing one or more conditions, diseases or disorders for which it is desirable to reduce fibrinogen and/or fibrin levels. In some examples, the inventors have discovered that lipid nanoparticles having lipid components as described herein and encapsulating siRNA targeting fibrinogen alpha chain mRNA could achieve controlled and/or sustained reduction of fibrinogen levels in the blood or other bodily sites. In further examples, controllably decreasing circulating fibrinogen and/or fibrin without compromising hemostasis through the use of such LNP composition could be used to safely decrease the concentration of fibrinogen in plasma for sustained periods of time.

According to one aspect of the disclosure, there is provided a lipid nanoparticle comprising: an siRNA molecule against fibrinogen alpha chain mRNA; an ionizable, cationic amino lipid having a pKa of between 5.5 and 7.0 and that is present at between 10 mol % and 85 mol %; a neutral, vesicle-forming lipid selected from at least one of a phospholipid and a triglyceride; a sterol; and a hydrophilic polymer-lipid conjugate present at between 0.5 mol % and 5 mol %.

According to one embodiment of the disclosure, the alpha chain of fibrinogen is human.

According to another example of any aspect or embodiment herein, the siRNA molecule comprises modified or unmodified nucleotides, and wherein the modified nucleotides are methylated.

According to another example of any aspect or embodiment herein, at least one strand of the duplex siRNA has a sequence that has at least 80% sequence identity to any one of SEQ ID NOs: 1 to 10 or 17-26.

In a further example of any aspect or embodiment herein, at least one strand of the duplex siRNA has a sequence that has at least 80% sequence identity to any one of SEQ ID NOs: 1 to 10 or 17-26.

According to another example of any aspect or embodiment herein, at least one strand of the duplex siRNA has a sequence that has at least 85% sequence identity to any one of SEQ ID NOs: 1 to 10 or 17-26.

According to a further example of any aspect or embodiment herein, at least one strand of the duplex siRNA has a sequence that has at least 90% sequence identity to any one of SEQ ID NOS: 1 to 10 or 17-26.

In a further example of any aspect or embodiment herein, at least one strand of the duplex siRNA has a sequence that has at least 95% sequence identity to any one of SEQ ID NOs: 1 to 10 or 17-26.

In a further example of any aspect or embodiment herein, the siRNA molecule is 15 to 35 nucleotides in length.

According to a further example of any aspect or embodiment herein, the siRNA molecule is 18 to 35 nucleotides in length.

According to a further example of any aspect or embodiment herein, the siRNA molecule is 20 to 30 nucleotides in length.

According to a further example of any aspect or embodiment herein, the siRNA molecule is a conjugate molecule. For example, the conjugate molecule may comprise a sugar group. In one embodiment, the sugar group comprises GalNAc.

According to a further aspect, the disclosure provides an siRNA molecule that has at least 80%, 85%, 90%, 95% or 97% sequence identity to any one of SEQ ID NOs: 1-10 or 17-26.

According to a further aspect, the disclosure provides an siRNA molecule that has at least 70%, 75%, 80%, 85%, 90%, 95% or 97% sequence identity to any one of SEQ ID NOs: 17-26.

According to a further aspect, the disclosure provides a pharmaceutical composition comprising the siRNA molecule or the lipid nanoparticle as described in any aspect or embodiment herein and wherein the pharmaceutical composition comprises a pharmaceutically acceptable salt and/or excipient.

According to a further example of any aspect or embodiment herein, after administration of the pharmaceutical composition to a patient, the patient's blood or plasma levels of fibrinogen does not fall below about 1 g/L (e.g., for up to 1 day to 3 weeks post-administration).

According to a further example of any aspect or embodiment herein, the pharmaceutical composition is for use to treat a fibrin (ogen)-dependent disorder in a patient in need of such treatment thereof.

According to a further example of any aspect or embodiment herein, there is provided a use of the pharmaceutical composition in the manufacture of a medicament to treat a fibrin (ogen)-dependent disorder.

According to a further example of any aspect or embodiment herein, there is provided a method of treating a patient having a fibrin (ogen)-dependent disorder comprising administering the pharmaceutical composition as described in any aspect of embodiment herein to a patient in need of such treatment thereof.

### BRIEF DESCRIPTION OF THE DRAWINGS

FIG. 1 shows human fibrinogen alpha chain (FGA) mRNA relative to control (%) for an empty lipid nanoparticle (LNP) control (Ctrl), and for LNP duplex siRNA sequences, hs.Ri.FGA.13.5 (duplex siRNA of SEQ ID Nos 1 and 2), CD.Ri.281933.13.5 (duplex siRNA of SEQ ID Nos 3 and 4), hs.Ri.FGA.13.8 (duplex siRNA of SEQ ID Nos 5 and 6) hs.Ri.FGA.13.4 (duplex siRNA of SEQ ID Nos 7 and 8), and hs.Ri.FGA.13.7 (duplex siRNA of SEQ ID Nos 9 and 10) (Table 1) after addition to HUH7 cells in vitro.

FIG. 2A shows fibrinogen alpha chain (Fga) mRNA relative to control (%) for a luciferase siRNA control (siLuc), and for duplex siRNA sequences, ms.FGA.1 (duplex siRNA of SEQ ID Nos 11 and 12), ms.FGA.2 (duplex siRNA of SEQ ID Nos 13 and 14) and ms.FGA.3 (duplex siRNA of SEQ ID Nos 15 and 16) (Table 2) siRNA one- and three-week post injection in mice.

FIG. 2B shows fibrinogen in plasma relative to control for duplex siLuc control and ms.FGA.1 (duplex siRNA of SEQ ID Nos 11 and 12), ms.FGA.2 (duplex siRNA of SEQ ID Nos 13 and 14) and ms.FGA.3 (duplex siRNA of SEQ ID Nos 15 and 16) (Table 2) siRNA one- and three-week post injection in mice.

FIG. 2C is a western blot detecting fibrinogen and platelet factor (PF4) in platelets from mice treated with control LNP siLuc and LNP siRNA targeting fibrinogen alpha chain ms.FGA.1 (duplex siRNA of SEQ ID Nos 11 and 12), ms.FGA.2 (duplex siRNA of SEQ ID Nos 13 and 14) and ms.FGA.3 (duplex siRNA of SEQ ID Nos 15 and 16) (Table 2).

FIG. 2D shows quantified signal intensities of fibrinogen and platelet factor 4 (PF4) bands on western blots detecting fibrinogen and PF4 in platelets from mice treated with control LNP siLuc and LNP siRNA targeting fibrinogen alpha chain ms.FGA.1 (duplex siRNA of SEQ ID Nos 11 and 12), ms.FGA.2 (duplex siRNA of SEQ ID Nos 13 and 14) and ms.FGA.3 (duplex siRNA of SEQ ID Nos 15 and 16) (Table 2).

FIG. 2E shows weekly fibrinogen levels in mouse plasma following injection with 0.1 mg/kg siRNA targeting fibrinogen alpha chain (siFga) corresponding to ms.FGA.2 (SEQ ID Nos 13 and 14) of Table 2.

FIG. 2F shows weekly fibrinogen levels in mouse plasma following injection with 0.5 mg/kg (top right) siRNA targeting fibrinogen alpha chain (siFga) corresponding to ms.FGA.2 (SEQ ID Nos 13 and 14) of Table 2.

FIG. 2G shows weekly fibrinogen levels in mouse plasma following injection with 1.0 mg/kg siRNA targeting fibrinogen alpha chain (siFga) corresponding to ms.FGA.2 (SEQ ID Nos 13 and 14) of Table 2.

FIG. 2H shows weekly fibrinogen levels in mouse plasma following injection with 2.0 mg/kg siRNA targeting fibrinogen alpha chain (siFga) corresponding to ms.FGA.2 (SEQ ID Nos 13 and 14) of Table 2.

FIG. 3A shows results of thromboelastography (TEG) in amplitude (mm) vs time (hours) to measure clot properties ex vivo in blood from mice treated with control siLuc (black) and siFga corresponding to ms.FGA.2 (SEQ ID Nos 13 and 14) of Table 2 (grey).

FIG. 3B shows clot time (min) of blood from mice treated with siRNA against luciferase (siLuc) (black) and siFga corresponding to ms.FGA.2 (SEQ ID Nos 13 and 14) of Table 2 (grey).

FIG. 3C shows rate of clot formation (angle) of blood from mice treated with siRNA against luciferase (siLuc) (black) and siFga corresponding to ms.FGA.2 (SEQ ID Nos 13 and 14) of Table 2 (grey).

FIG. 3D shows clot stiffness (mm) of blood from mice treated with siRNA against luciferase (siLuc) (black) and siFga corresponding to siFga corresponding to ms.FGA.2 (SEQ ID Nos 13 and 14) of Table 2 (grey).

FIG. 3E shows bleed time (min; left graph) and blood loss (μL/g; right graph) after saphenous vein puncture in mice treated with siRNA against luciferase (siLuc) and siFga corresponding to ms.FGA.2 (SEQ ID Nos 13 and 14) of Table 2 (grey).

FIG. 3F shows bleed time (min; left graph) and blood loss (μL/g; right graph) after tail transection in mice treated with siRNA against luciferase (siLuc) and siFga corresponding to ms.FGA.2 (SEQ ID Nos 13 and 14) of Table 2.

FIG. 4A shows fibrinogen levels in plasma from mice treated with siLuc (black) and siFga corresponding to ms.FGA.2 (SEQ ID Nos 13 and 14) of Table 2 (grey) right before inducing inferior vena cava (IVC) stasis.

FIG. 4B shows weight of thrombus formed in the IVC of mice treated with siLuc (black) and and siFga corresponding to ms.FGA.2 (SEQ ID Nos 13 and 14) of Table 2 (grey).

FIG. 5A shows fibrinogen alpha chain (Fga) mRNA levels in mouse livers collected at the endpoint of a lipopolysaccharide (LPS)-induced endotoxemia study. Mice were treated first with siLuc (black circles) and siFga corresponding to ms.FGA.2 (SEQ ID Nos 13 and 14) (grey diamonds), then phosphate buffered saline (PBS) vehicle control and LPS.

FIG. 5B shows fibrinogen beta chain (Fgb) mRNA levels in mouse livers collected at the endpoint of a lipopolysaccharide (LPS)-induced endotoxemia study. Mice were treated first with siLuc (black circles) and siFga corresponding to ms.FGA.2 (SEQ ID Nos 13 and 14) (grey diamonds), then phosphate buffered saline (PBS) vehicle control and LPS.

FIG. 5C shows fibrinogen gamma chain (Fgg) mRNA levels in mouse livers collected at the endpoint of a lipopolysaccharide (LPS)-induced endotoxemia study. Mice were treated first with siLuc (black circles) and siFga corresponding to ms.FGA.2 (SEQ ID Nos 13 and 14) (grey diamonds), then phosphate buffered saline (PBS) vehicle control and LPS.

FIG. 5D shows fibrinogen levels in mouse plasma collected at the endpoint of a lipopolysaccharide (LPS)-induced endotoxemia study. Mice were treated first with siLuc (black circles) and siFga corresponding to ms.FGA.2 (SEQ ID Nos 13 and 14) (grey diamonds), then phosphate buffered saline (PBS) vehicle control and LPS.

FIG. 5E shows D-dimer levels in mouse plasma, representing activated coagulation, collected at the endpoint of a lipopolysaccharide (LPS)-induced endotoxemia study. Mice were treated first with siLuc (black circles) and siFga corresponding to ms.FGA.2 (SEQ ID Nos 13 and 14) (grey diamonds), then phosphate buffered saline (PBS) vehicle control and LPS.

FIG. 5F shows levels of the inflammatory cytokines, tumour necrosis factor-α (TNFα), interleukin-1β, (IL-1b), IL-17, monocyte chemoattractant protein-1 (MCP-1), and macrophage inflammatory protein-1α (MIP-1α) in mouse plasma, collected at the endpoint of a lipopolysaccharide (LPS)-induced endotoxemia study. Mice were treated first with siLuc (black bars) and siFga corresponding to ms.FGA.2 (SEQ ID Nos 13 and 14) (grey bars), then phosphate buffered saline (PBS) vehicle control and LPS.

FIG. 6A shows fibrinogen levels in mouse plasma collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were administered thioglycollate.

FIG. 6B shows total leukocyte cell count in mouse peritoneal lavage fluid collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were administered thioglycollate.

FIG. 6C shows monocyte/macrophage cell count in mouse peritoneal lavage fluid collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were administered thioglycollate.

FIG. 6D shows T cell count in mouse peritoneal lavage fluid collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were administered thioglycollate.

FIG. 6E shows B cell count in mouse peritoneal lavage fluid collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were administered thioglycollate.

FIG. 6F shows neutrophil cell count in mouse peritoneal lavage fluid collected at the endpoint of a thioglycollate-induced peritonitis study. Plasminogen knockout (Plg^{-/-}) mice were treated with siLuc (light grey) and siFga (dark grey) corresponding to ms.FGA.2 (SEQ ID Nos 13 and 14) of Table 2. All mice, including plasminogen sufficient (Plg^{+/+}; black) mice for comparison, were given administered thioglycollate.

FIG. 7A shows fibrinogen levels in plasma of mice treated with siLuc (black) and siFga (grey) corresponding to ms.FGA.2 (SEQ ID Nos 13 and 14) of Table 2, collected at endpoint of an experimental metastasis study.

FIG. 7B shows total number of pulmonary foci in lungs of mice treated with siLuc (black) and siFga (grey) corresponding to ms.FGA.2 (SEQ ID Nos 13 and 14) of Table 2, collected at the endpoint of an experimental metastasis study.

FIG. 7C shows the number of macrometastases (left graph) and micrometastases (right) in lungs of mice treated with siLuc (black) and siFga (grey) corresponding to ms.FGA.2 (SEQ ID Nos 13 and 14) of Table 2, collected at endpoint of an experimental metastasis study.

FIG. 7D shows image of macrometastases (white large arrowhead) and micrometastases (small white arrow) in lung lobes of mice treated with siLuc (left image) and siFga (right image) corresponding to ms.FGA.2 (SEQ ID Nos 13 and 14) of Table 2, collected at the endpoint of an experimental metastasis study.

### DETAILED DESCRIPTION

Throughout this specification, unless the context requires otherwise, the words “comprise”, “comprising” and the like, are to be construed in an inclusive sense as opposed to an exclusive sense, that is to say, in the sense of “including, but not limited to”.

One embodiment of the disclosure provides a lipid nanoparticle comprising an siRNA sequence to reduce the expression of the alpha chain of fibrinogen. Fibrinogen has three chains, namely alpha, beta and gamma chains. In some embodiments, mRNA encoding the alpha chain is targeted by the siRNA sequence and thereby reduces or prevents the assembly of the fibrinogen protein by the liver. In some embodiments, this in turn reduces secretion of fibrinogen into the blood. In certain advantageous embodiments, the inventors have found that the LNP formulations as described herein can control circulating fibrinogen levels within a range that does not compromise hemostasis (e.g., above a threshold of 1 g/L in blood or serum of a patient). This is particularly advantageous in that the composition of the disclosure can address safety concerns of previous methods for reducing fibrinogen levels.

The siRNA described herein may modulate the levels of one or both of fibrinogen and fibrin (the latter term also referred to herein as “fibrin (ogen)”). As would be known by those of skill in the art, fibrinogen can be cleaved post-translation to fibrin. Therefore, by targeting the mRNA encoding the alpha chain of fibrinogen, the siRNA may reduce the levels of fibrinogen and/or fibrin in a bodily site.

The siRNA targeting the alpha chain of fibrinogen is a duplex siRNA. In such embodiment, the siRNA comprises a sense strand and an antisense strand, each nucleotide of the siRNA being a modified or unmodified nucleotide, and the sense and antisense strands having at least partial complementarity. Further non-limiting examples of the disclosure are described in more detail hereinafter.

siRNA

The siRNA described herein is used to treat, ameliorate, or prevent a “fibrinogen-dependent condition, disease or disorder”. The term encompasses, in some examples, conditions, diseases or disorders resulting from elevation of fibrinogen above a normal level. In alternative examples, it may be desirable to reduce fibrinogen below a normal level. Examples of fibrin (ogen)-dependent diseases or disorders, include, but are not limited to: hyperfibrinogenemia, acute inflammation after microbial and/or viral infections (e.g., sepsis, COVID-19), chronic inflammation (e.g., associated with increasing age and in diseases such as obesity, arthritis, diabetes, and the like); thrombosis, including thrombosis associated with cancer and trauma; thromboinflammation (thrombosis associated with increased inflammation); cardiovascular disease; and cancer growth, progression, and metastasis.

The expression “siRNA molecule against fibrinogen alpha chain mRNA” as used herein includes a double-stranded RNA (i.e., duplex RNA such as siRNA, aiRNA, or pre-miRNA) that reduces or inhibits the expression of fibrinogen such as by mediating the degradation or inhibiting the translation of an mRNA that is complementary to the siRNA sequence as measured in vitro or in vivo. The siRNA may have substantial or complete identity to the gene that encodes a fibrinogen alpha chain or sequence, or may comprise a region of mismatch (i.e., a mismatch motif). The sequence of the siRNA can correspond to the full-length target sequence, or a subsequence thereof.

In some embodiments, the siRNA is 15 to 40 or 20 to 35 nucleotides in length. Since the siRNA is double-stranded, the nucleotide length corresponds to the length of the shorter of an antisense or sense strand.

The siRNA described herein may comprise a “mismatch motif” or “mismatch region”, which refers to a portion of the siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

In some embodiments, the siRNA reduces or inhibits expression of fibrinogen as measured in vitro or in vivo. Inhibition or reduction of expression of fibrinogen is achieved when reduction of MRNA obtained with an siRNA relative to a relevant control (e.g., buffer or an empty lipid nanoparticle) is about 90%, 85%, 80%, 75%, 70%, 65%, 60%, 55%, 50%, 45%, 40%, 35%, 30%, 25%, 20%, 15%, 10%, 5% or 0%. Suitable assays for measuring expression of a target gene or target sequence include, e.g., examination of protein or RNA levels using techniques known to those of skill in the art such as quantitative PCR (qPCR), western blots, dot blots, northern blots, in situ hybridization, ELISA, immunoprecipitation, enzyme function, as well as phenotypic assays known to those of skill in the art. The reduction in expression in vitro may be measured using an assay as described in the Example section. Phenotypic assays include clotting or other assays in model organisms as described herein in the Example section to assess treatment or prevention of a fibrinogen-dependent disease.

The expression “inhibiting or reducing expression of fibrinogen”, includes inhibition or reduction of fibrinogen alpha chain expression that is achieved when the value obtained with an interfering RNA relative to a relevant control is about 90%, 85%, 80%, 75%, 70%, 65%, 60%, 55%, 50%, 45%, 40%, 35%, 30%, 25%, 20%, 15%, 10%, 5% or 0% using any one of the assays set forth above. Either mRNA or protein levels may be assayed in certain embodiments.

The nucleotides of the siRNA may be modified. Examples of modifications include, but are not limited to, 2′-O-alkyl modifications such as 2′-O-Me modifications and 2′-halogen modifications such as 2′-fluoro modifications.

The siRNA may have sequence identity to any one of the nucleotide sequences set forth in Table 1, Table 2 and Table 3 below. More typically, the siRNA has sequence identity to the human nucleotide sequences set forth in Table 1 or Table 3. The expression “sequence identity” when referring to two nucleic acids herein, refers to two sequences or subsequences that are the same or have a specified percentage of nucleotides that are the same, when compared and aligned for maximum correspondence over a comparison window, or designated region as measured using a known comparison algorithm or by manual alignment and visual inspection.

For determining sequence identity, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. The sequence identity is typically measured by BLAST, which is well-known to those of skill in the art.

In one embodiment, the siRNA has at least 30% to 100% sequence identity to any one of SEQ ID NOs: 1-26 in Table 1, Table 2 and Table 3 below. For example, the siRNA may have at least 40%, at least 50%, at least 55%, at least 60%, at least 65%, at least 70%, at least 75%, at least 80%, at least 85%, at least 90%, at least 95%, at least 98% sequence identity to any one of SEQ ID Nos: 1-26. In one embodiment, a strand of the siRNA consists essentially of any one of SEQ ID NOS: 1-26 meaning that the strand differs by no more than 4 nucleotides but excluding modifications of the nucleotides, such as methylation or a halogen modification (described below).

In a further embodiment, the siRNA or a strand of a duplex siRNA differs by no more than 1, 2, 3, 4, 5, 7, 8, 9, 10, 11, 12, 13, 14 or 15 nucleotides as set forth in SEQ ID NOs: 1-26. In one embodiment, the siRNA differs by no more than 10 nucleotides or no more than 5 nucleotides. In in one embodiment, this excludes differences due to modifications of a given nucleotide, such as methylation or a halogen modification (described below).

In another embodiment the present disclosure provides one or more exemplary siRNA sequences or duplexes thereof selected from SEQ ID NOs: 1-10 or SEQ ID NOs: 17-26 (human sequences) to inhibit or reduce the expression of fibrinogen.

In one embodiment, the siRNA has at least 30% to 100% sequence identity to any one of SEQ ID NOs: 1-10 in Table 1 or SEQ ID NOs 17-26 in Table 3 below. For example, the siRNA may have at least 40%, at least 50%, at least 55%, at least 60%, at least 65%, at least 70%, at least 75%, at least 80%, at least 85%, at least 90%, at least 95%, at least 98% sequence identity to any one of SEQ ID Nos: 1-10 in Table 1 or SEQ ID NOs 17-26 in Table 3 below. In one embodiment, the siRNA consists essentially of any one of SEQ ID NOs: 1-10 in Table 1 or SEQ ID NOs: 17-26 in Table 3 below, meaning that it differs by no more than 4 nucleotides excluding modifications of the nucleotides, such as methylation or a halogen modification (described below).

It should be appreciated that the sequence identity herein need not require an exact match of two nucleotides. To illustrate, a given nucleotide can be methylated and will be considered to have identity to an unmethylated nucleotide.

In a further embodiment, the siRNA or a strand of a duplex siRNA differs by no more than 1, 2, 3, 4, 5, 7, 8, 9, 10, 11, 12, 13, 14 or 15 nucleotides as set forth in SEQ ID NOs: 1-10 in Table 1 and SEQ ID NOs: 17-26 in Table 3 below. In one embodiment, the siRNA differs by no more than 10 nucleotides or no more than 8, 7, 6 or 5 nucleotides from the sequences in Table 1 and Table 3 below. In in one embodiment, this excludes differences due to modifications of a given nucleotide, such as methylation or a halogen modification (described below).

In another embodiment the present disclosure provides one or more siRNA sequences or duplexes thereof selected from SEQ ID NOs: 1-10 (Table 1) and SEQ ID NOs: 17-26 (Table 3) to inhibit or reduce the expression of fibrinogen.

In another embodiment, the present disclosure provides one or more siRNA sequences or duplexes thereof selected from SEQ ID NOs: 1-10 (Table 1) to inhibit or reduce the expression of fibrinogen and the siRNA or a strand of a duplex siRNA differs by no more than 1, 2, 3, 4, 5, 7, 8, 9, 10, 11, 12, 13, 14 or 15 nucleotides and/or 0 to 50% or 10 to 40% of the nucleotides have 2′-O-alkyl modifications such as 2′-O-Me modifications and/or 2′-halogen modifications.

Without being limiting, the siRNA sequences may exhibit a modification pattern similar to that set forth in Table 2 or Table 3 below.

TABLE 1
Base composition of duplex siRNA sequence
targeting human fibrinogen alpha chain mRNA.

| | | Base composition of the |
|---|---|---|
| | SEQ | duplex siRNA targeting |
| | ID | human fibrinogen alpha |
| Sequence Name | NO. | chain mRNA |
| hs.Ri.FGA.13.5 | 1 | 5′-GCUCUGUAUCUGGUAGUACUGG |
| | | ACA-3′ |
| | 2 | 5′-UGUCCAGUACUACCAGAUACAG |
| | | AGCUC-3′ |
| CD.Ri.281933.13.5 | 3 | 5′-CUGAUGGUCACAAAGAAGUUAC |
| | | CAA-3′ |
| | 4 | 5′-UUGGUAACUUCUUUGUGACCAU |
| | | CAGGA-3′ |
| hs.Ri.FGA.13.8 | 5 | 5′-AGGGUUGAUUGAUGAAGUCAAU |
| | | CAA-3′ |
| | 6 | 5′-UUGAUUGACUUCAUCAAUCAAC |
| | | CCUUU-3′ |
| hs.Ri.FGA.13.4 | 7 | 5′-GGUGGACAUUGAUAUUAAGAUC |
| | | CGA-3′ |
| | 8 | 5′-UCGGAUCUUAAUAUCAAUGUCC |
| | | ACCUC-3′ |
| hs.Ri.FGA.13.7 | 9 | 5′-AUAGUGGUGAAGGUGACUUUCU |
| | | AGC-3′ |
| | 10 | 5′-GCUAGAAAGUCACCUUCACCAC |
| | | UAUCU-3′ |

TABLE 2
Base modification of duplex siRNA sequences targeting murine
fibrinogen alpha chain mRNA, “r” designates unmodified base,
“m” designates 2′O-methylated base

| | SEQ | |
|---|---|---|
| Sequence | ID | Base composition of the duplex siRNA targeting |
| Name | NO | murine fibrinogen alpha chain mRNA (5′-3′) |
| ms.FGA.1 | 11 | mCmArAmCrCmArGrGrArUrUmUrUmArCmArAmArCrArGrArAmUC |
| | 12 | rGrAmUrUrCrUrGrUmUrUmGrUmArArArArUrCrCrUrGrGmUrUm |
| | | GmGmC |
| ms.FGA.2 | 13 | mGmCrUmGrUmArArArCrCrGmUrGmArGmArUmArArArUrCrUmAC |
| | 14 | rGrUmArGrArUrUrUmArUmCrUmCrArCrGrGrUrUrUrArCmArGm |
| | | CmCmC |
| ms.FGA.3 | 15 | mCmArGmGrUmCrArUrCrGrCmUrAmArAmGrAmArUrUrGrCrUmUC |
| | 16 | rGrAmArGrCrArArUmUrCmUrUmUrArGrCrGrArUrGrArCmCrUm |
| | | GmUmU |

TABLE 3
Human siRNA sequences with modifications. “r” designates
unmodified base, “m” designates 2′O-methylated base

| | SEQ | |
|---|---|---|
| | ID | Base composition of the duplex siRNA targeting |
| Sequence Name | NO | human fibrinogen alpha chain mRNA (5′-3′) |
| hs.Ri.FGA.13.5 | 17 | mGmCrUmCrUmGrUrArUrCrUmGrGmUrAmGrUmArCrUrGrGrAmC |
| | | rA |
| methylated | 18 | rUrGmUrCrCrArGrUmArCmUrAmCrCrArGrArUrArCrArGmArG |
| | | mCmUmC |
| CD.Ri.281933.13.5 | 19 | mCmUrGmArUmGrGrUrCrArCmArAmArGmArAmGrUrUrArCrCmA |
| | | rA |
| methylated | 20 | rUrUmGrGrUrArArCmUrUmCrUmUrUrGrUrGrArCrCrArUmCrA |
| | | mGmGmA |
| hs.Ri.FGA.13.8 | 21 | mAmGrGmGrUmUrGrArUrUrGmArUmGrAmArGmUrCrArArUrCmA |
| | | rA |
| methylated | 22 | rUrUmGrArUrUrGrAmCrUmUrCmArUrCrArArUrCrArArCmCrC |
| | | mUmUmU |
| hs.Ri.FGA.13.4 | 23 | mGmGrUmGrGmArCrArUrUrGmArUmArUmUrAmArGrArUrCrCmG |
| | | rA |
| methylated | 24 | rUrCmGrGrArUrCrUmUrAmArUmArUrCrArArUrGrUrCrCmArC |
| | | mCmUmC |
| hs.Ri.FGA.13.7 | 25 | mAmUrAmGrUmGrGrUrGrArAmGrGmUrGmArCmUrUrUrCrUrAmG |
| | | rC |
| methylated | 26 | rGrCmUrArGrArArAmGrUmCrAmCrCrUrUrCrArCrCrArCmUrA |
| | | mUmCmU |

It should be appreciated that an siRNA having a sequence similar to those set forth in the sequence listings may optionally be conjugated with another moiety, such as but not limited to a ligand, as described below.

Within an siRNA, the antisense strand and the sense strand may be designed such that when they form a duplex due to complementarity of base-pairs, they can anneal with no overhangs and thus form blunt ends at both ends of the duplex, or with an overhang at one or more of the 3′ end of the sense strand, the 3′ end the antisense strand, the 5′ end of the sense strand, and the S′ end of the antisense strand. In some embodiments, there are no 5′ overhangs and there is no 3′ antisense overhang, but there is a 3′ sense overhang. In other aspects, there are no 5′ overhangs, but there are a 3′ antisense overhang and a 3′ sense overhang.

When overhangs are present, they may, for example, be 1 to 6 nucleotides long. In some aspects, the overhang is a dinucleotide. By way of a non-limiting example, in one aspect, there is a 3′ sense overhang that is dTdT, and there are no overhangs on the antisense strand and no 5′ sense overhang. By way of another non-limiting example, in another aspect, there are a 3′ sense overhang that is dTdT and a 3′ antisense overhang that also is dTdT, but there are no 5′ overhangs on either the antisense strand or the sense strand. By way of another non-limiting example, in one aspect, there is a 3′ sense overhang that is dTdT, and a 3′ dinucleotide antisense overhang that is complementary to two nucleotides on the target molecule adjacent to the region of the target molecule to which the region of the antisense strand within the duplex is complementary. In this aspect, there are no 5′ overhangs on either the antisense strand or the sense strand. When an overhang is present, the nucleotides within it are included in the aforementioned range of 18 to 30 nucleotides for each strand.

In some aspects, the siRNA are covalently bound to one or more other molecules to form a conjugate. In some aspects, the conjugates are selected because they facilitate delivery of the siRNA to an organism or into cells. An siRNA may be bound to a conjugate at, for example, the 5′ end of the antisense strand, the 3′ end of the antisense strand, the 5′ end of the sense strand, the 3′ end of the sense strand, or to a nucleotide at a position that is not at the 3′ end or 5′ end of either strand.

Examples of conjugates include but are not limited to one or more of an antibody, a peptide, an amino acid, an aptamer, a phosphate group, a cholesterol moiety, a lipid, a cell-penetrating peptide polymer, and a sugar group, which includes a sugar monomer, an oligosaccharide and modifications thereof. In one aspect, the conjugate is N-Acetylgalactosamine (GalNAc).

### Lipid Nanoparticles

In one embodiment, the disclosure provides a nucleic acid against fibrinogen alpha chain mRNA that is encapsulated within a lipid nanoparticle. In one embodiment, the nucleic acid is for inhibiting or reducing expression of fibrinogen.

It will be understood that the invention is not limited by the location or the nature of the incorporation of the nucleic acid within the lipid nanoparticle. That is, the term “encapsulated” is not meant to be limited to any specific interaction between the nucleic acid and the lipid nanoparticle. The nucleic acid may be incorporated in the aqueous portion, within any lipid layer or both.

The lipid nanoparticle (LNP) described herein may comprise an ionizable lipid that may associate or complex with the nucleic acid. The term “ionizable lipid” refers to any of a number of lipid species that carry a net positive charge at a selected pH below its pKa. In some embodiments, the cationic lipid has a head group comprising an amino group. In some embodiments, the cationic lipids comprise a protonatable tertiary amine (e.g., pH titratable) head group, C16 to C18 alkyl chains, ether linkages between the head group and alkyl chains, and 0 to 3 double bonds.

In certain embodiments, the cationic lipid content is from 20 mol % to 70 mol % or 30 mol % to 55 mol % or 35 mol % to 55 mol % of total lipid present in the lipid nanoparticle.

The lipid nanoparticle (LNP) described herein may comprise a helper lipid in addition to the ionizable lipid. In the context of the present disclosure, the term “helper lipid” includes any vesicle-forming lipid (e.g., bilayer-forming lipid) that may be selected from a phosphatidylcholine lipid, sphingomyelin, or mixtures thereof. In some embodiments, the helper lipid is selected from sphingomyelin, distearoylphosphatidylcholine (DSPC), dioleoylphosphatidylcholine (DOPC), 1-palmitoyl-2-oleoyl-phosphatidylcholine (POPC) and dipalmitoyl-phosphatidylcholine (DPPC). In certain embodiments, the helper lipid is DOPC, DSPC or sphingomyelin. In one embodiment, the helper lipid is DSPC. The helper lipid content may include mixtures of two or more different types of different helper lipids.

For example, in certain embodiments, the phosphatidylcholine content is from 20 mol % to 60 mol % or 25 mol % to 60 mol % or 30 mol % to 60 mol % or 35 mol % to 60 mol % or 40 mol % to 60 mol % of total lipid present in the lipid nanoparticle. The phosphatidylcholine lipid content is determined based on the total amount of lipid in the lipid nanoparticle, including the sterol.

In one embodiment, the LNP comprises a sterol, a hydrophilic polymer-lipid conjugate or both.

Examples of sterols include cholesterol, or a cholesterol derivative, such as cholestanol, cholestanone, cholestenone, coprostanol, cholesteryl-2′-hydroxyethyl ether, cholesteryl-4′-hydroxybutyl ether, beta-sitosterol, fucosterol and the like. In one embodiment, the sterol is present at from 15 mol % to 65 mol %, 18 mol % to 50 mol %, 20 mol % to 50 mol %. 25 mol % to 50 mol % or 30 mol % to 50 mol % based on the total lipid present in the lipid nanoparticle. In another embodiment, the sterol is cholesterol and is present at from 15 mol % to 65 mol %, 18 mol % to 50 mol %, 20 mol % to 50 mol %, 25 mol % to 50 mol % or 30 mol % to 50 mol % based on the total lipid and sterol present in the lipid nanoparticle.

In one embodiment, the hydrophilic-polymer lipid conjugate includes (i) a vesicle-forming lipid having a polar head group, and (ii) covalently attached to the head group, a polymer chain that is hydrophilic. Example of hydrophilic polymers include polyethyleneglycol (PEG), polyvinylpyrrolidone, polyvinylmethylether, polyhydroxypropyl methacrylate, polyhydroxypropylmethacrylamide, polyhyd.-oxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate.

The hydrophilic polymer lipid conjugate may be present in the nanoparticle at 0 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In another embodiment, the PEG-lipid conjugate is present in the nanoparticle at 0 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

### Methods to Treat or Prevent Fibrin (Ogen)-Dependent Diseases

In another aspect, the present disclosure provides methods of treating a subject having any disorder or condition that would benefit from a reduction in fibrinogen expression.

This includes a “inflammatory disorder”, which as used herein includes any condition, of any severity, that results in abnormal amounts of leukocytes, inflammatory cytokines/chemokines, or acute phase proteins, such as but not limited to fibrinogen, in a subject. The fibrin (ogen)-dependent disease or disorder includes but is not limited to COVID-19, cancer, sepsis, obesity, microbial/viral infections, and thrombosis. The methods include administering to the subject a therapeutically effective amount of the siRNA, optionally encapsulated in a lipid nanoparticle, thereby treating the subject or providing a prophylactic effect

As used herein, the term “subject” includes any human or non-human mammalian subject that would benefit from a reduction in fibrinogen expression relative to lack of treatment thereof. This includes a prophylactic benefit in some embodiments. In some embodiments, the subject is a human.

In one embodiment, the disclosure provides methods of preventing at least one symptom, e.g., thrombosis, in a subject having a disorder that would benefit from reduction in fibrinogen expression. The methods include administering to the subject a therapeutically effective amount of the siRNA, thereby preventing at least one symptom in the subject having a disorder that would benefit from reduction in fibrinogen expression.

In one embodiment, the administration of the siRNA to the subject causes a decrease in thrombosis, inflammation, and/or a decrease in fibrinogen protein expression and/or accumulation.

In another embodiment the present disclosure provides a method of treating a patient by modulating coagulation, the method comprising: administering siRNA to a subject in need thereof to inhibit the expression of fibrinogen. Modulation of coagulation or clotting can be assessed as set forth in the Example section herein.

Further methods for assessing knockdown, inhibition and/or reduction in fibrinogen expression include thromboelastography (TEG), a clot stiffness assay, a clot lysis assay and/or quantifying plasma fibrinogen protein concentration. Inhibition of expression of a target gene or target sequence is achieved when the value obtained with an siRNA relative to a relevant control is about 90%, 85%, 80%, 75%, 70%, 65%, 60%, 55%, 50%, 45%, 40%, 35%, 30%, 25%, 20%, 15%, 10%, 5%, or 0%.

In another embodiment, the siRNA is used to treat a cell in vitro or in vivo. The cell may be within a subject, such as a mammalian subject, for example a human subject suffering from a fibrin (ogen)-dependent disorder. One embodiment of the disclosure provides a method to knock-down fibrinogen using siRNA delivered to hepatocytes.

### Pharmaceutical Formulations

In some embodiments, the siRNA or lipid nanoparticle comprising a nucleic acid reducing expression fibrinogen is part of a pharmaceutical composition and is administered to treat and/or prevent a disease condition. The treatment may provide a prophylactic (preventive), ameliorative or a therapeutic benefit to treat fibrinogen-dependent disease. The pharmaceutical composition will be administered at any suitable dosage.

In one embodiment, the pharmaceutical composition is administered parenterally, i.e., intra-arterially, intravenously, subcutaneously or intramuscularly. In another embodiment, the pharmaceutical compositions are administered intranasally, intravitreally, subretinally, intrathecally or via other local routes.

The pharmaceutical composition comprises pharmaceutically acceptable salts and/or excipients. Used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

As used herein, the term “excipient” means the substances used to formulate active pharmaceutical ingredients (API) into pharmaceutical formulations. Non-limiting examples include mannitol, Captisol®, lactose, starch, magnesium stearate, sodium saccharine, talcum, cellulose, sodium crosscarmellose, glucose, gelatin, sucrose, magnesium carbonate, and the like. Acceptable excipients are non-toxic and may be any solid, liquid, semi-solid excipient that is generally available to one of skill in the art.

The examples are intended to illustrate preparations and properties of the invention but are in no way intended to limit the scope of the invention.

### EXAMPLES

### Materials and Methods

siRNA-LNP Preparation, Analysis, and Administration

Three mouse-specific and five human-specific siRNA sequences targeting different regions of Fga mRNA (siFga) for each species were designed in silico. siRNA was dissolved in 25 mM sodium acetate pH 4 buffer at an amine-to-phosphate (N/P) ratio of 3. Lipids ionizable amino lipid, DSPC, cholesterol and PEG-DMG, were dissolved in ethanol at a molar ratio of 50/10/38.5/1.5 mol %, respectively, to achieve a final concentration of 20 mM total lipid. The two solutions were mixed using a T-junction mixer as described previously, and the resulting siRNA-lipid nanoparticles (LNPs) were dialyzed against phosphate buffered saline (PBS) pH 7.4 in a 500-fold excess. Cholesterol content was measured using a Cholesterol E Assay Kit (Wako Chemicals, Mountain View, CA), from which total lipid concentration was extrapolated. Nucleic acid entrapment was determined using a RiboGreen™ assay. siRNA-LNPs were administered to mice and dogs intravenously. siFga was used as treatment, and PBS or siRNA targeting luciferase (siLuc) were used as a control for mouse studies. For screening human siFga sequences, empty LNPs were used as control. When siRNA-LNPs were used within a week of formulation, they were stored at 4° C. and dilutions were made with phosphate-buffered saline (PBS). If longer storage was needed, dilutions were made with a buffer containing 10 mM L-histidine and 10% sucrose (pH 7.4) and stored at −80° C. until ready-to-use. The siRNA-LNPs were administered at the indicated doses.

### Mice

Procedures performed at each institution were approved by the local Animal Care Committee Wild-type (WT) C57BL/6J mice (stock #000664; The Jackson Laboratory) age 8-14 weeks were used unless otherwise indicated.

### Murine Blood Draws

Blood samples was drawn from isoflurane anesthetized mice by retroorbital sampling for non-terminal blood draw or cardiac puncture for terminal blood draw, using a 23G needle containing sodium citrate (0.32% or 0.38% final) to a final v/v concentration of 10% in whole blood. Whole blood was centrifuged at 1,500×g for 10 min twice to obtained platelet-poor plasma. To isolate platelets, 300 mL of Tyrode's buffer (pH 6.5) was added to whole blood, then centrifuged at 600×g for 3 min to obtain platelet-rich plasma (PRP). Prostaglandin E1 (10 mg/ml, Sigma) was added to PRP and all subsequent spin steps to minimize platelet activation. PRP was centrifuged again 400×g for 2 min to remove remaining red blood cells (RBC). PRP was subsequently centrifuged at 800×g for 10 min to isolate the pelleted platelets.

mRNA Quantification

Unless otherwise indicated, cells were collected from culture plates, or liver tissue was surgically removed from anesthetized mice and was homogenized in Trizol (ThermoFisher, Waltham, MA). Nucleic acid was extracted by phenol-chloroform precipitation. DNA was digested by incubating the sample with TURBO DNase (ThermoFisher) at 37° C. for 1 hour. DNase was removed by repeating the Trizol-chloroform extraction. Reverse transcription was performed using the iScript cDNA Synthesis Kit (Bio-Rad, Hercules, CA) followed by qPCR with SYBR Green Master Mix (ThermoFisher) and DNA primers (IDT, Coralville, IA).

### Protein Quantification

Fibrinogen protein levels were quantified by enzyme-linked immunosorbent assay (ELISA) (Innovative Research and Enzyme Research Laboratories), or western blot. For western blot, samples were reduced, boiled, and separated on 4-15% acrylamide gradient gels (Bio-Rad). After electrophoresis, the samples were transferred to a nitrocellulose membrane (GE Healthcare) and blocked with Odyssey Blocking Buffer (LI-COR). The membrane was incubated with rabbit anti-human fibrinogen antibody (1:10,000; A0080, Agilent Dako), or platelet factor 4 (PF4, 1:1000; SAPF4-AP; Affinity Biologicals). After incubating with HRP-conjugated goat anti-rabbit secondary antibody (1:20,000; ab7090; Abcam), specific bands were detected using ECL substrate (Bio-Rad) using a Sapphire Biomolecular Imager (Azure Biosystems). Quantification of fibrinogen bands was performed using ImageJ software and expressed as relative intensity to the PF4 loading control.

### Thromboelastography (TEG)

Shear elastic moduli were evaluated at 37° C. using a TEG Hemostasis Analyzer System 5000 (Haemonetics). Citrated mouse whole blood was mixed with a calcium-saline buffer (50 mM CaCl2 and 90 mM NaCl) and recombinant tissue factor (Innovin, 10 pM, MedCorp).

Screen siFga in Human Hepatoma (HUH7) Cell Culture

HUH7 cells were seeded at 1×10⁵ cells/well one day prior to transfection. Cells were then transfected with the five different LNP-hsiFga at a dose of 3 μg/mL of siRNA, or empty LNPs as a negative control. The following day, cells were lysed, and the RNA was extracted using the PureLink RNA Mini Kit (Thermo Fisher, Waltham, MA) following the manufacturer's protocol. Fibrinogen mRNA levels was then detected and quantified using SYBR Green qPCR as described above.

### Saphenous Vein Puncture Bleeding Model

One weeks after siFga (1 mg/kg) or PBS administration, mice were anesthetized with 10-15% isoflurane, and kept on a heating pad. After fur removal, vein was visualized under 10× magnification stereoscope. Vein was isolated from artery and nerve; after a rest period of approximately 5 minutes, a puncture wound was made in the medial wall of the saphenous vein using the bevel of a 23-gauge needle. Blood loss over time was measured by gently absorbing blood at the puncture site with pre-weighed filter paper until bleeding stopped. Bleeding was monitored for 40 minutes. Blood loss was measured by weight of paper after blood absorption.

### Tail Transection Bleeding Model

One weeks after siFga or siLuc (both 1 mg/kg) administration, mice were anesthetized with 10-15% isoflurane, and kept on a heating pad. Tail tails were transected four mm from the tip and immediately immersed in a 0.9% NaCl solution (saline) to monitor bleeding for 20 minutes. To quantify blood loss, the blood-saline samples were treated with an RBC lysis solution (1.5 M NH4Cl, 0.1 M NaHCO3, 0.01 M EDTA), incubated at room temperature for 10 minutes while gently shaking, then absorbance measured at 509 nm (Tecan microplate reader). The absorbance was converted to amount of blood loss, using a standard curve with known amounts of mouse blood collected by intracardiac puncture, and normalized to body weight.

### Inferior Vena Cava (IVC) Stasis Model of Thrombosis

Mice were injected with siFga or siLuc (2 mg/kg) via tail vein 6 days prior to inducing IVC stasis. The IVC was exposed, isolated and ligated. Side branches were also ligated, and lumbar branches were cauterized. After 24 hours, thrombi were removed from the IVC and weighed.

### Endotoxemia Model

Mice were injected with siFga or siLuc (1 mg/kg) via tail vein one-week prior to intraperitoneal injection with 10 mg/kg of lipopolysaccharide (LPS, Sigma). Mice were euthanized 24-hours after LPS injection, and blood and livers were collected for analysis. Hepatic mRNA expression levels of Fga, BB chain (Fgb), and y chain (Fgg) were determined using TaqMan gene expression assays (Applied Biosystems) on an ABI StepOne Plus sequence detection system (Applied Biosystems). The expression of each gene was normalized relative to B2m expression levels, and relative expression level determined using the Pfaffl method. Plasma levels of fibrinogen, and D-dimer were quantified by ELISA (Immunology Consultants Laboratory INC, Siemens Healthcare Diagnostics and Diagnostica Stago, respectively). Cytokines in platelet poor plasma, including TNFα, IL-1β, IL-17, MCP-1, and MIP-1 α, were measured using a multiplex cytokine analysis by the Advanced Analytics Core at UNC Chapel Hill.

### Thioglycollate-Induced Peritonitis Model

Plasminogen-deficient (Plg^{-/-}) mice were injected with siFga or siLuc (1 mg/kg) one-week prior to inducing peritonitis. Plasminogen sufficient (Plg^{+/+}) mice were not treated with siRNA prior to peritonitis. Peritonitis was induced by intraperitoneal injection of 500 μL 4% Brewer thioglycollate medium (BD Difco). Seventy-two hours after challenge, the peritoneal cavity was lavaged with 5 mL of PBS. Lavage fluid was analyzed by differential cell count and flow cytometry by a blinded investigator as previously described. Blood was collected, and plasma fibrinogen levels were quantified as described above.

### Experimental Metastasis Model

Mice were injected with siFga or siLuc (2 mg/kg) at week-3, -2, -1 and day 0 via tail vein prior to tumour cell inoculation. Thirty minutes after the last siRNA-LNP injection, mice were injected with 300 μL of GFP-expressing Lewis Lung Carcinoma (LLCGFP) cells (3.0×10\hat{ }5 cells) via tail vein, then euthanized on day 14. The cells were grown in complete media (DMEM, 10% FBS, 2 mM L-Glut, 2% Pen/Strep) for at least one passage and reached 70% confluency. The cells were harvested by brief exposure to trypsin/EDTA, washed, and resuspended in ice-cold PBS. Fourteen days after tumour inoculation, lungs were harvested and pulmonary LLCGFP foci were counted by a blinded investigator using a fluorescent microscope. Blood was collected from a subset of mice 14 days after challenge and plasma fibrinogen levels were determined as described above.

### Statistical Analysis

A Shapiro-Wilkes test was performed to determine whether data were normally distributed. Pairwise comparisons were performed with two-tailed unpaired Student's t test or Mann-Whitney U test. Comparisons between multiple groups with one variable were performed by regular one-way analysis of variance (ANOVA) followed by Tukey's multiple comparison test for normally distributed and unpaired data. Data not normally distributed were compared by Kruskal-Wallis (unpaired) or Friedman (paired) test, followed by Dunn's multiple comparison tests. Two-way ANOVA followed by Tukey's multiple comparison test was used to compare datasets with two variables. All statistical comparisons were performed with Graphpad Prism 9 (Graphpad Software, U.S.A.). For cytokine datasets, values that were out of range of the standard curves were treated as partially observed values (censored observation approach) using R to obtain imputed values.

### Example 1: SiRNA Knock Down In Vitro

This example demonstrates that siRNA knocks down fibrinogen in human hepatocytes in vitro.

Quantitative PCR as described in the Materials and Methods was used to measure human fibrinogen alpha chain (FGA) mRNA levels after administration of different siRNA sequences human fibrinogen alpha chain mRNA (siFga) to human hepatocyte cells in culture.

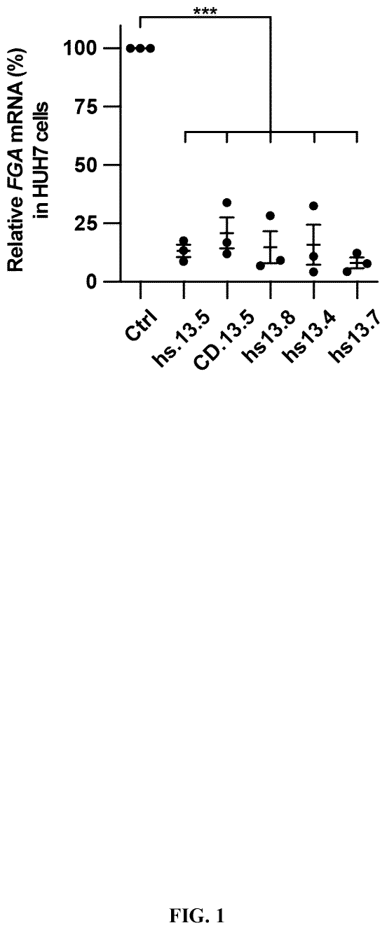

As shown in FIG. 1, significant depletion of FGA mRNA was observed in vitro after treatment with LNP containing the siRNA sequences set out in Table 1, namely hs.Ri.FGA.13.5 (hs.13.5) (SEQ ID Nos 1 and 2), CD.Ri.281933.13.5 (CD.13.5) (SEQ ID Nos 3 and 4), hs.Ri.FGA.13.8 (hs.13.8) (SEQ ID Nos 5 and 6), hs.Ri.FGA.13.4 (hs.13.4) (SEQ ID Nos 7 and 8), and hs.Ri.FGA.13.7 (hs.13.7) (SEQ ID Nos 9 and 10), compared to empty LNPs as negative control (ctrl) in human HUH7 cells.

### Example 2: SiRNA Knock Down of Fibrinogen In Vivo

This example demonstrates that siRNA knocks down fibrinogen alpha chain mRNA in mice, resulting in depletion of circulating fibrinogen protein in vivo.

PCR (qPCR) as described in the Materials and Methods was used to quantify hepatic fibrinogen alpha chain mRNA levels after administration of three different murine siRNA sequences targeting fibrinogen to mice 1 and 3 weeks prior to tissue collection, and compared to control siRNA targeting luciferase (siLuc).

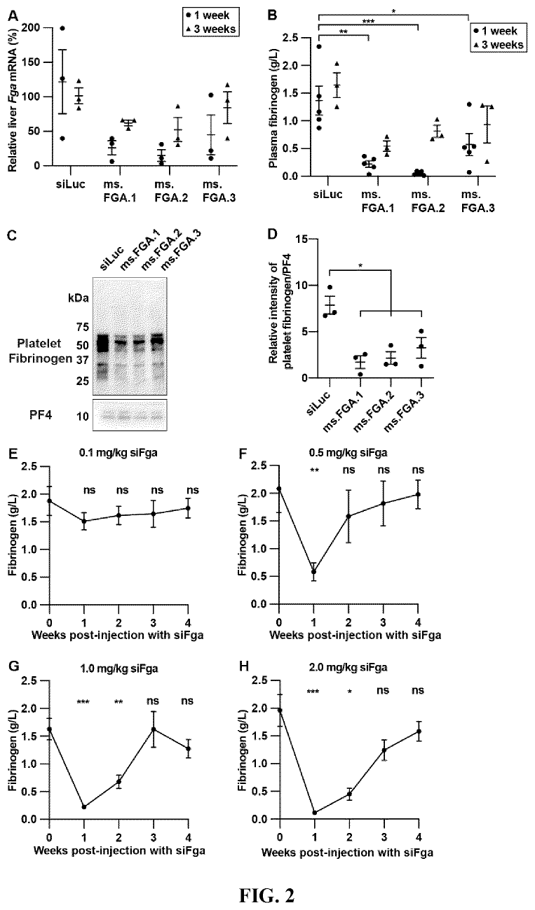

The results are shown in FIG. 2A. As can be seen in FIG. 2A, hepatic fibrinogen mRNA relative to control (siLuc) was reduced in the liver tissue of mice by 1 week after administration of the following siRNA sequences: ms.FGA.1 (SEQ ID Nos 11 and 12), ms.FGA.2 (SEQ ID Nos 13 and 14) and ms.FGA.3 (SEQ ID Nos 15 and 16) of Table 2.

ELISA as described in the Materials and Methods was used to quantify fibrinogen protein levels in blood plasma after administration of three different murine siRNA sequences targeting fibrinogen alpha chain mRNA to mice 1 and 3 weeks prior to blood sampling, and compared to siLuc.

The results are shown in FIG. 2B. As can be seen in FIG. 2B, compared to control siLuc, fibrinogen protein levels were significantly reduced in the blood plasma of mice by 1 week after administration of the following siRNA sequences: ms.FGA.1 (SEQ ID Nos 11 and 12), ms.FGA.2 (SEQ ID Nos 13 and 14) and ms.FGA.3 (SEQ ID Nos 15 and 16) of Table 2.

Western blot as described in the Materials and Methods was used to quantify fibrinogen protein levels in platelets after administration of three different murine siRNA sequences targeting fibrinogen alpha chain mRNA to mice 1 week prior to blood sampling, and compared to siLuc.

The results are shown in FIG. 2C and FIG. 2D. A representative western blot is shown in FIG. 2C and the signal intensities of fibrinogen and platelet factor 4 (PF4; loading control) bands representing their relative abundance was quantified and shown in FIG. 2D. Compared to control siLuc, fibrinogen protein levels were significantly reduced in the platelets of mice by 1 week after administration of the following siRNA sequences: ms.FGA.1 (SEQ ID Nos 11 and 12), ms.FGA.2 (SEQ ID Nos 13 and 14) and ms.FGA.3 (SEQ ID Nos 15 and 16) of Table 2.

As described in the Material and Methods, a dosing study was performed and ELISA was used to quantify fibrinogen protein levels in blood plasma weekly for 4 weeks after administration of siFga corresponding to ms.FGA.2 (SEQ ID Nos 13 and 14) of Table 2 at a single dose at 0.1, 0.5, 1.0, and 2.0 mg/kg. Fibrinogen levels at each week were quantified and compared to baseline fibrinogen levels, using blood collected 3 days prior to injection with siFga.

The results are shown in FIG. 2E-H. Administering increasingly higher dose of siFga corresponding to ms.FGA.2 (SEQ ID Nos 13 and 14) of Table 2 led to increased depletion of fibrinogen protein from blood plasma at 1 week post-administration, with a slower rate of recovery back to baseline.

### Example 3: SiRNA Knock Down of Fibrinogen and Effects on Clotting Ex Vivo and In Vivo

This example shows that siRNA knocks down fibrinogen alpha chain in mice impairs clotting ex vivo, but does not impair clot formation in vivo following injuries.

Thromboelastography (TEG) as described in the Materials and Methods was used to measure clot properties ex vivo in blood from mice treated with siLuc (black) and siFga corresponding to ms.FGA.2 (SEQ ID Nos 13 and 14) of Table 2 (grey). Clot time, rate of clot formation, and clot stiffness were quantified from TEG.

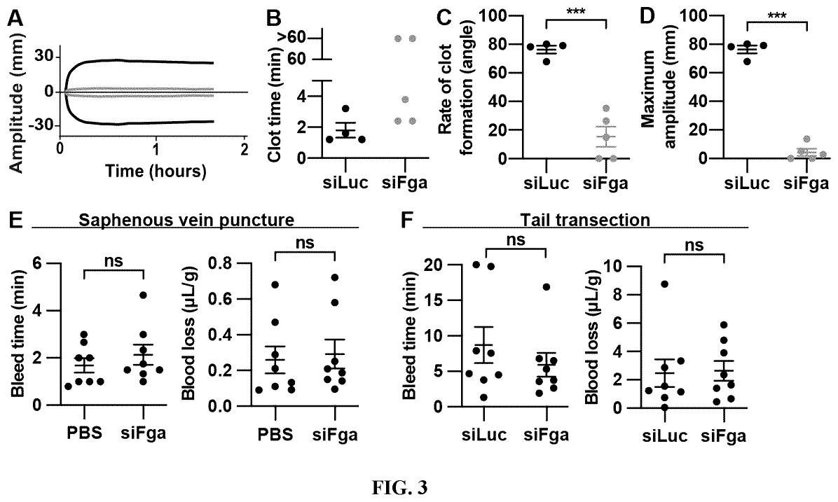

FIG. 3 shows a representative thromboelatography curve. Blood from mice treated with siFga corresponding to ms.FGA.2 (SEQ ID Nos 13 and 14) of Table 2 (grey) have significantly impaired clotting compared to blood from siLuc-treated mice (black).

FIG. 3B compares time to clot formation in blood from mice treated with siLuc and siFga corresponding to ms.FGA.2 (SEQ ID Nos 13 and 14) of Table 2 (grey). Two siFga-treated mice did not clot throughout the duration of the assay, while blood from all 5 siLuc-treated mice formed a clot.

FIG. 3C compares the rate of clot formation in blood from mice treated with siLuc and siFga corresponding to ms.FGA.2 (SEQ ID Nos 13 and 14) of Table 2 (grey), showing that blood from siFga-treated mice formed a clot slower than blood from siLuc-treated mice.

FIG. 3D compares clot stiffness in blood from mice treated with siLuc and siFga corresponding to ms.FGA.2 (SEQ ID Nos 13 and 14) of Table 2 (grey), showing that blood from siFga-treated mice forms a significantly weaker clot compared siLuc-treated mice.

For bleeding models, mice were administered with siFga corresponding to ms.FGA.2 (SEQ ID Nos 13 and 14) of Table 2 and PBS or siLuc as negative control one week prior to inducing injury. A saphenous vein puncture or tail transection was performed, and bleeding and blood loss was monitored over time as described in the Materials and Methods.

FIG. 3E shows the bleed time and blood loss of mice following saphenous vein puncture. As can be seen in FIG. 3E, mice treated with siFga corresponding to ms.FGA.2 (SEQ ID Nos 13 and 14) of Table 2 bled for similar duration and have comparable blood loss compared to control PBS-treated mice.

FIG. 3F shows the bleed time and blood loss of mice following tail transection. As can be seen in FIG. 3E, mice treated with siFga corresponding to ms.FGA.2 (SEQ ID Nos 13 and 14) of Table 2 bled for similar duration and have comparable blood loss, compared to control siLuc-treated mice.

### Example 4: Depletion of Plasma Fibrinogen with siRNA Decreases Thrombosis

This example shows siRNA knockdown of fibrinogen alpha chain can decrease thrombosis in vivo.

Mice were administered siLuc (black) or siFga corresponding to ms.FGA.2 (SEQ ID Nos 13 and 14) of Table 2 (grey). Blood was collected for plasma fibrinogen quantification prior to inducing inferior vena cava (IVC) stasis as described in the Material and Methods.

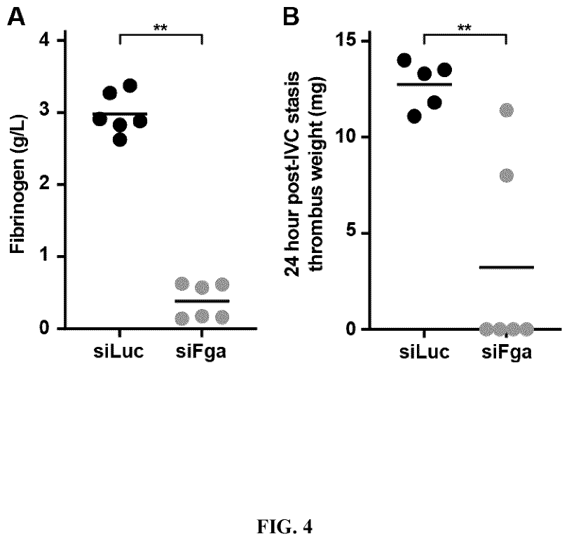

As shown in FIG. 4A, fibrinogen protein level was significantly depleted prior to inducing IVC stasis. As shown in FIG. 4B, siFga-treated mice have significantly smaller thrombus formed in the IVC compared to siLuc-treated mice.

### Example 5: Depletion of Plasma Fibrinogen with siRNA Attenuates Lipopolysaccharide (LPS)-Induced Acute Phase Response

This example demonstrates that siRNA knockdown of fibrinogen attenuates the acute phase response in a model of endotoxemia.

Mice were administered siLuc (black) or siFga corresponding to ms.FGA.2 (SEQ ID Nos 13 and 14) of Table 2 (grey) prior to inducing endotoxemia with lipopolysaccharide (LPS) injection, as described in the Material and Methods.

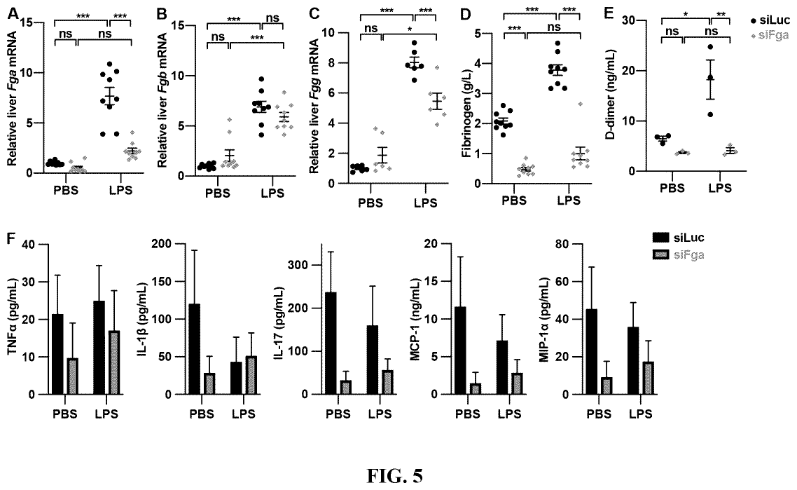

Hepatic levels of the fibrinogen alpha chain (Fga), beta chain (Fgb), and gamma chain (Fgg) were quantified as described in the Material and Methods, and results are shown in FIGS. 5A, 5B, and 5C, respectively. As shown in these figures, LPS challenge led to significantly increased levels of each Fga, Fgb and Fgg mRNA levels in siLuc pre-treated mice, and pre-treatment with siFga attenuated this increase for Fga and Fgg mRNA, but not Fgb mRNA.

Plasma fibrinogen, D-dimer, and cytokine levels, including tumour necrosis factor-α (TNFα), interleukin-1β, (IL-1β), IL-17, monocyte chemoattractant protein-1 (MCP-1), and macrophage inflammatory protein-la (MIP-la), were quantified as described in the Material and Methods, and results are shown in FIGS. 5D, 5E and 5F, respectively.

As shown in FIG. 5D, LPS-induced upregulation of plasma fibrinogen was attenuated by siFga pre-treatment.

As shown in FIG. 5E, LPS-induced upregulation of plasma D-dimer was attenuated by siFga pre-treatment.

As shown in FIG. 5F, siFga pre-treatment led to lower levels of each cytokine in the blood plasma of both LPS-treated and PBS (negative control) treated mice, except for IL-1B in the LPS-treated mice.

### Example 6: Depletion of Plasma Fibrinogen with siRNA Restores Impaired Macrophage Migration in Plasminogen Deficient Mice

This example shows that impaired of macrophage migration in plasminogen deficient (Plg^{-/-}) mice can be rescued by depletion of plasma fibrinogen with siRNA targeting fibrinogen alpha chain.

Plg^{-/-} mice were administered siLuc (light grey) or siFga corresponding to ms.FGA.2 (SEQ ID Nos 13 and 14) of Table 2 (dark grey) prior to inducing peritonitis with thioglycollate as described in the Material and Methods. Plasminogen sufficient mice (Plg^{+/+}) mice were also used as a control in the study.

ELISA as described in the Materials and Methods was used to quantify fibrinogen protein levels in blood plasma at study endpoint.

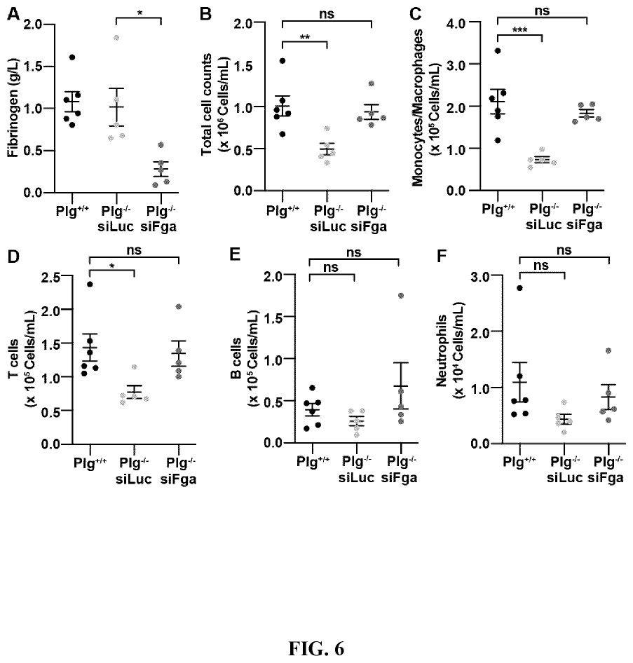

The results are shown in FIG. 6A. Fibrinogen was significantly depleted in blood plasma of siFga-treated Plg^{-/-} mice compared to siLuc-treated Plg^{-/-} mice

Peritoneal lavage fluid was collected at endpoint, and leukocyte count in was quantified as described in the Material and Methods.

FIG. 6B shows total leukocyte count in the peritoneal lavage fluid, and the levels of the specific types of leukocytes, including monocyte/macrophages, T cells, B cells, and neutrophils are shown in FIGS. 6C, 6D, 6E, and 6F, respectively.

As shown in FIGS. 6B and 6C, siLuc-treated Plg^{-/-} mice had significantly less leukocytes in the peritoneal lavage fluid, driven primarily by significant decrease in monocyte/macrophages, compared to Plg^{+/+} mice. Treatment with siFga prior to inducing peritonitis restored the number of leukocytes recruited to the peritoneal cavity to numbers comparable to those in Plg^{+/+} mice.

FIG. 6D shows that siLuc-treated Plg^{-/-} mice also had significantly less T cells in the peritoneal lavage fluid compared to Plg^{+/+} mice, while treatment with siFga prior to inducing peritonitis restored the number of T cells in the peritoneal lavage fluid to numbers comparable to those in Plg^{+/+} mice.

FIGS. 6E and 6F shows that neither the B cells and neutrophil counts do not differ in siLuc- or siFga-treated Plg^{-/-} mice compared to Plg^{+/+} mice.

### Example 7: Depletion of Plasma Fibrinogen with siRNA Decreases Metastatic Potential of Tumour Cells

This example shows that fibrinogen knockdown with siRNA decreases cancer metastasis in vivo. Mice were administered siLuc (black) or siFga corresponding to ms.FGA.2 (SEQ ID Nos 13 and 14) of Table 2 (grey) prior to injecting fluorescent tumour cells as described in the Material and Methods.

ELISA as described in the Materials and Methods was used to quantify fibrinogen protein levels in blood plasma at study endpoint.

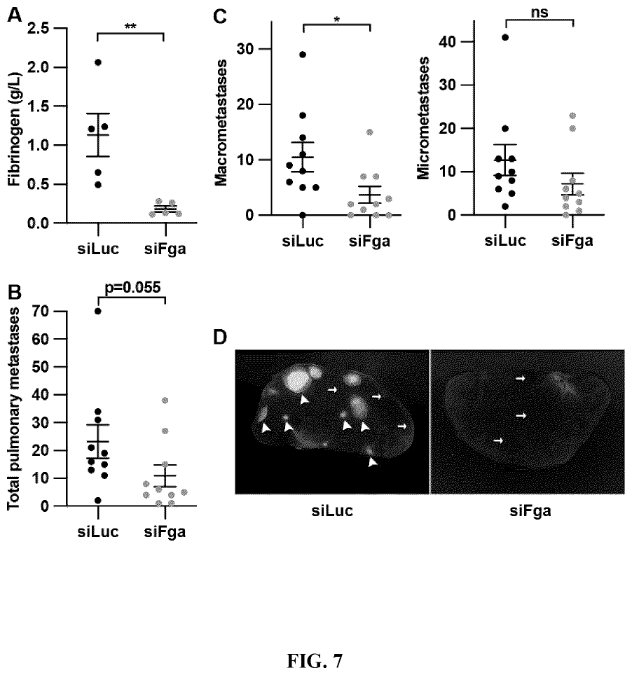

FIG. 7A shows fibrinogen is significantly depleted in blood plasma of siFga-treated mice at endpoint compared to siLuc-treated mice.

The number of pulmonary metastases was counted as described in the Material and Methods.

FIG. 7B shows that there is a trend towards decreased number of total metastases in the lungs in siFga-treated mice.

FIG. 7C shows that there was significantly less macrometastases formed in the lungs of siFga-treated mice compared to siLuc-treated mice, while the number micrometastases in the lungs of siFga-treated mice was comparable to siLuc-treated mice.

FIG. 7D shows a representative image of lung lobes from each of siLuc- and siFga-treated mice, with both macrometastases (large white arrowheads) and micrometastases (small white arrows) evident in the siLuc-treated mice but only micrometastases evident in siFga-treated mice.

Although the invention has been described and illustrated with reference to the foregoing detailed description and examples, it will be apparent that a variety of modifications and changes may be made without departing from the invention.

1. A lipid nanoparticle comprising:
an siRNA molecule against fibrinogen alpha chain mRNA;
an ionizable, cationic amino lipid having a pKa of between 5.5 and 7.0 and that is present at between 10 mol % and 85 mol %;
a neutral, vesicle-forming lipid selected from at least one of a phospholipid and a triglyceride;
a sterol; and
a hydrophilic polymer-lipid conjugate present at between 0.5 mol % and 5 mol %.

2. The lipid nanoparticle of claim 1, wherein the alpha chain of fibrinogen is human.

3. The lipid nanoparticle of claim 1, wherein the siRNA molecule comprises modified or unmodified nucleotides, and wherein the modified nucleotides are methylated.

4. The lipid nanoparticle of claim 1, wherein at least one strand of the duplex siRNA has a sequence that has at least 80% sequence identity to any one of SEQ ID NOs: 1 to 10 or 17-26.

5. The lipid nanoparticle of claim 1, wherein at least one strand of the duplex siRNA has a sequence that has at least 80% sequence identity to any one of SEQ ID NOs: 1 to 10 or 17-26.

6. The lipid nanoparticle of claim 1, wherein at least one strand of the duplex siRNA has a sequence that has at least 85% sequence identity to any one of SEQ ID NOs: 1 to 10 or 17-26.

7. The lipid nanoparticle of claim 1, wherein at least one strand of the duplex siRNA has a sequence that has at least 90% sequence identity to any one of SEQ ID NOs: 1 to 10 or 17-26.

8. The lipid nanoparticle of claim 1, wherein at least one strand of the duplex siRNA has a sequence that has at least 95% sequence identity to any one of SEQ ID NOs: 1 to 10 or 17-26.

9. The lipid nanoparticle of claim 1, wherein the siRNA molecule is 15 to 35 nucleotides in length.

10. The lipid nanoparticle of claim 9, wherein the siRNA molecule is 18 to 35 nucleotides in length.

11. The lipid nanoparticle of claim 9, wherein the siRNA molecule is 20 to 30 nucleotides in length.

12. The lipid nanoparticle of claim 1, wherein the siRNA molecule is a conjugate molecule.

13. The lipid nanoparticle of claim 12, wherein the conjugate molecule comprises a sugar group.

14. The lipid nanoparticle of claim 13, wherein the sugar group comprises GalNAc.

15. An siRNA molecule having at least 80% sequence identity to any one of SEQ ID NOs: 1-10 or 17-26.

16. The siRNA molecule of claim 15 having at least 85% sequence identity to any one of SEQ ID NOs: 1-10 or 17-16.

17. The siRNA molecule of claim 15 having at least 90% sequence identity to any one of SEQ ID NOs: 1-10 or 17-26.

18. The siRNA molecule of claim 15 having at least 95% sequence identity to any one of SEQ ID NOs: 1-10 or 17-26.

19. A pharmaceutical composition comprising the siRNA molecule of claim 15, and wherein the pharmaceutical composition comprises a pharmaceutically acceptable salt and/or excipient.

20-22. (canceled)

23. A method of treating a patient having a fibrin (ogen)-dependent disorder comprising administering the pharmaceutical composition of claim 19 to a patient in need of such treatment thereof.
